(19) United States
(12) Patent Application Publication
(10) Pub. No.: US 2003/0183963 A1
(43) Pub. Date: Oct. 2, 2003

(54) Ceramic material for dental applications and a method for the production thereof

(76) Inventor: Wolfgang Wiedemann, Hochberg (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/297,419

(22) PCT Filed: Jun. 6, 2001
(86) PCT No.: PCT/EP01/06401

(30) Foreign Application Priority Data
Jun. 8, 2000 (DE) 100-27-976.5

(51) Int. Cl. B29B 9/00; C04B 35/00

(52) U.S. Cl. 264/6; 501/1; 106/35

(57) ABSTRACT

The invention relates to a dental ceramic comprising a sintered body that has a share of more than 90% by weight hydroxylapatite (HA; Ca₅(PO₄)₃OH). The ceramic is easy to produce and highly stable, and in addition offers a natural-looking appearance if the sintered body is anisotropic.

Ceramic material for use in dental applications along with a process for manufacturing said material, and the use of a starting material from the production process for dental applications.

The present invention relates to a ceramic material for use in dental applications, especially in fillings and dentures. The invention further relates to a process for manufacturing a material of this type, and the use of a starting material from the manufacturing process for dental applications.

It has long been known that human and animal tooth enamel is comprised essentially of hydroxylapatite (Ca₅(PO₄)₃(OH)). Over time various methods have been developed by which a synthetic form of hydroxylapatite may be produced, which is suitable for use in dental applications, especially as inlay material or in dentures.

Several times hydroxylapatite combined with certain additives has been suggested for use as a ceramic in dentures. For example, in DE 3935060 it is proposed that readily soluble calcium phosphates, such as monetite or brushite, be added to the hydroxylapatite.

From DE 19614016 a process is known, in which a diphosphate or a polyphosphate is added to the aqueous phase, prior to the precipitation of the hydroxylapatite. This leads to an addition of tricalcium phosphate to the hydroxylapatite in the final product.

Finally, as the most recent state of the art, U.S. Pat. No. 4,097,935 is known, in which substantially pure hydroxylapatite is proposed for use as a dental ceramic material. The hydroxylapatite ceramic disclosed therein is isotropic in its physical properties, and in optical terms is not doubly refractive.

All dental prosthetic ceramics in accordance with the above-described state of the art are biologically compatible, and generally display adequate stability in the oral cavity in terms of their chemical properties. It is nonetheless considered disadvantageous that these ceramic materials are not translucent. Thus in a pure state they are pure white in appearance, in a raw state they resemble chalk, and when polished they resemble very white porcelain. Coloration of these materials is possible only to a limited extent. It is thus not possible to produce natural-looking tooth colors.

It is thus the object of the present invention to provide a dental prosthetic ceramic, a process for manufacturing said dental prosthetic ceramic, and a starting substance for use in dental applications, which, in addition to the essential properties required of natural tooth enamel, will also offer an appearance that more closely resembles natural tooth enamel.

This object is attained with a ceramic having the characteristics specified in claim 1. The object is further attained with a process having the characteristic features specified in claim 7.

Because the sintered body is anisotropic, the lattice planes of the crystallites that form the sintered body are oriented opposite to a preferred direction. This results in a decrease in the internal reflection in the sintered body. The sintered body itself thus becomes somewhat translucent, causing it to resemble natural tooth enamel.

If the refraction index is anisotropic within the spectrum of visible light, and especially if the sintered body exhibits double refraction, then the optical properties of the sintered body lie within the preferred range. Hence, a particularly natural appearance is offered by a difference in the refractive index of Δn≧1 10⁻⁴, especially Δn≧2 10⁻³. With this type of double refraction, the color of the material that lies beneath the tooth enamel is essential to the tooth color. Thus it can be set substantially higher than the color of the cement beneath it. The sintered body is preferably also anisotropic in terms of x-ray diffraction, wherein the intensity of reflection can be altered by texture, in other words by preferred orientations within the sintered body. This type of anisotropy is advantageous because with it a formed double refraction (caused by scattering, elliptical cavities filled with air, for example) can be excluded, in favor of an intrinsic double refraction caused by textured effects.

In this manner the optical properties are improved. Finally, it is advantageous if the anisotropy is oriented toward a specific axis, for example the axis of symmetry of a cylindrical ceramic body. When this is the case, the properties of the sintered body are better defined, for example, in terms of mechanical workability.

An advantageous sintered body is one in which the content of tricalcium phosphate (TCP) and/or another poorly soluble phosphate is ≦4%. This also contributes to a low level of opacity and stability inside the oral cavity for the material.

Because in the process specified in the invention the Ca/P atomic ratio lies between 1.66 and 1.68, the number of optically effective scattering centers in the sintered body is low, which serves to decrease opacity. The calcium phosphate compound, which is precipitated via the process specified in the invention, advantageously is substantially stoichiometric hydroxylapatite.

The pressing of the green body is preferably accomplished at an intrinsic pressure of 200 bar to 10,000 bar, preferably from 800 bar to 1,500 bar. The latter range produces a favorable ratio of optical properties for the sintered body and economic feasibility of the manufacturing process. For a cylindrical green body, the pressing is preferably performed in an axial direction. The optical properties can be further improved if the pressing is performed via an extrusion die in an axial direction, with the extrusion die being rotated around its axis.

The object is further attained with a dental ceramic that is produced in accordance with the process specified in claims 7-11.

Using a fine-crystalline hydroxylapatite as the starting material for dental applications enables the creation of dental ceramics that exhibit the desired properties, as long as the individual crystallites are rod-shaped and between 10 nm and 1,000 nm long, and between 5 nm and 500 nm thick.

Finally, the object is attained with the use of a crystalline hydroxylapatite in accordance with claim 13 to produce a dental ceramic for use in treating dental diseases.

Below, three exemplary embodiments of the present invention will be described with respect to their synthesis, with the help of tables and diagrams. These show:

Table 1: the half-intensity width of the lines of a calcium phosphate precipitated in accordance with Example 1, in an x-ray diffraction diagram;

Table 2: the intensities of the reflections in the x-ray diffraction diagram of the sintered body in Example 1;

Table 3: the intensities of the reflections in the x-ray diffraction diagram of the sintered body in Example 2;

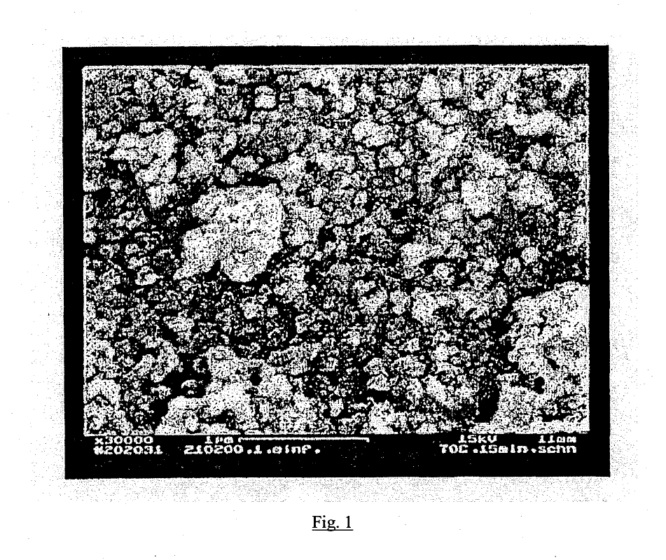

FIG. 1: the precipitation product produced in Example 1, enlarged approximately 30,000 times;

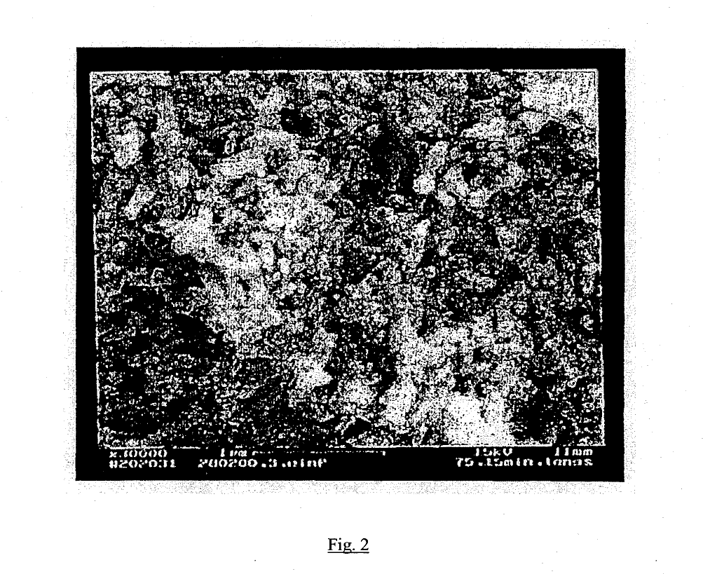

FIG. 2: the precipitation product produced in Example 2, enlarged approximately 30,000 times; and

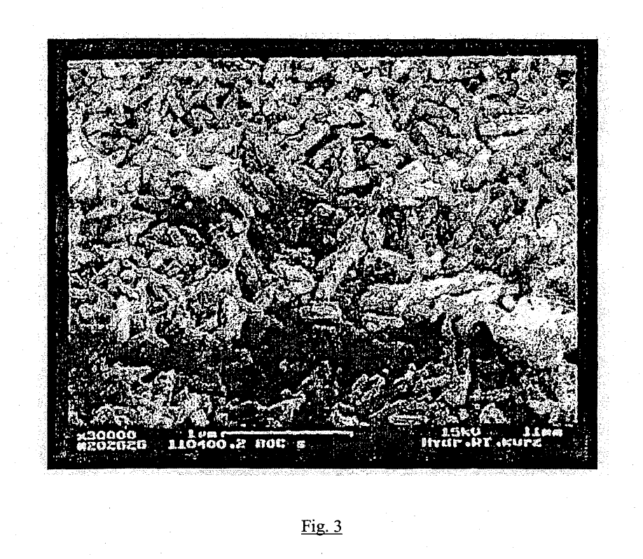

FIG. 3: the precipitation product produced in Example 3, enlarged approximately 30,000 times.

### EXAMPLE 1

153 g Ca(NO₃)_{2.4}H₂O are dissolved in 1 l aqua bidest (18 MΩ cm). 250 ml of this are drawn off and mixed with 44 g NH₃ (32%). 17.33 g (NH₄)₂HPO₄ are dissolved in 1 l aqua bidest (18 MΩ/cm). 750 ml of this are drawn off and mixed with 8.8 g NH₃ (32%). All chemicals used possess the purity level p.a. To the receiving flask, 1.1 l aqua bidest, 3 ml of the Ca solution, and 8.8 g NH₃ (32%) are added, and the contents are heated to 70° C.

The reaction takes place in an external reaction vessel that has a volume of ca. 5 ml, a throughput rate of ca. 200 ml/s, and a stirring speed of 400/s, with high shear forces at a constant temperature. The Ca solution is added to the receiving flask dropwise, at a rate of 0.33 ml/s. The phosphate solution is introduced into the external reaction vessel at a rate of 0.77 ml/s.

Upon completion of the reaction, the precipitate is allowed to rest on the mother liquor for 18 h at room temperature, after which it is washed with room temperature aqua bidest until the nitrate level in the rinsing water is <5 ppm. Following filtration and drying at 210° C., a yield of 14.12 g of precipitate is obtained.

The precipitate is a calcium phosphate having the lattice structure of apatite. Both wet chemical tests and the x-ray diffraction spectrum after being heated to more than 900° C. point to stoichiometric hydroxylapatite.

The precipitate is comprised of quite fluffy, needle-like particles, ca. 150 nm in length and 50 nm in width, as is illustrated in FIG. 1. The line width of the (002) reflection in the x-ray diffraction diagram is significantly smaller than the reflection of lattice planes that lie parallel to the c axis, see Table 1.

For further processing, the precipitate is ground in an agate mortar to particles that are <250 μm, is axially pressed at 2400 bar, and is then sintered using the following time/temperature profile: room temperature up to 400° C.: 13° C./min; stationary 400° C.: 60 min; 400° C. to 850° C.: 10° C./min; stationary 850° C.: 120 min; 850° C. to 1195° C.: 3° C./min; stationary 1195° C.: 60 min; cooling to room temperature: ca. 1.5° C./min.

The green body displays an intrinsic double refraction of Δn=(2.0±0.5)*10⁻³ with the “quick axis” being perpendicular to the pressing direction.

As a result of the sintering, we obtain a translucent body having a thickness of 3.15 g/cm³. The double refraction was calculated as Δn=(0.82±0.11)*10⁻³, with the c-axis being perpendicular to the pressing direction. The x-ray diffraction diagram indicates that the sintered body is pure hydroxylapatite. The anisotropy is also apparent in the x-ray diffraction diagram. The intensities of the reflections are indicated in Table 2. The relative intensity indicates the measured intensity of the given line as a percentage of the intensity of the (211) reflection. In the “isotropy” column, the relative intensities of the reflections for pulverized samples are indicated, in accordance with the JCPDS. The “orientation” column indicates the approximate orientation of a given lattice plane relative to the c-axis.

### EXAMPLE 2

153 g Ca(NO₃)_{2.4}H₂O are dissolved in 1 l aqua bidest (18 MΩ/cm). 250 ml of this are drawn off and mixed with 44 g NH₃ (32%). 17.33 g (NH₄)₂HPO₄ are dissolved in 1 l aqua bidest (18 MΩ/cm). 750 ml of this are drawn off and mixed with 8.8 g NH₃ (32%). All chemicals used possess the purity level p.a. To the receiving flask, 1.1 l aqua bidest, 3 ml of the Ca solution, and 8.8 g NH₃ (32%) are added, and the contents are heated to 75° C.

The reaction takes place in an external reaction vessel that has a volume of ca. 5 ml, a throughput rate of ca. 78 ml/s, and a stirring speed of 160/s, at a constant temperature, over a period of 16 min. The Ca solution is added to the receiving flask dropwise, at a rate of ca. 0.32 ml/s. The phosphate solution is introduced into the external reaction vessel at a rate of 0.63 ml/s.

Upon completion of the reaction, the precipitate is allowed to stand 18 h at room temperature, after which it is washed with room temperature aqua bidest until the nitrate level in the rinsing water is <5 ppm. Following filtration and drying at 210° C., a yield of 13.25 g precipitate is obtained. The relatively fluffy precipitate is comprised of crystalline rods, which are ca. 250 nm long and 50 nm thick, see FIG. 2.

For further processing, the precipitate is ground in an agate mortar to particles that are <250 μm, is axially pressed at 800 bar, and is then sintered using the following time/temperature profile: room temperature up to 400° C.: 13° C./min; stationary 400° C.: 60 min; 400° C. to 850° C.: 10° C./min; stationary 850° C.: 120 min; 850° C. to 1195° C.: 3° C./min; stationary 1195° C.: 60 min; cooling to room temperature: ca. 1.5° C./min.

The green body displays an intrinsic double refraction of Δn=(1.4±0.7)*10⁻³ with the “quick axis” being perpendicular to the pressing direction. The result of the sintering is a translucent body having a thickness of 3.14 g/cm³. The double refraction was calculated as Δn=(1.2±0.1)*10⁻³, with the c-axis being perpendicular to the pressing direction. The x-ray diffraction diagram indicates that the sintered body is pure hydroxylapatite. The anisotropy is also apparent in the x-ray diffraction diagram. The intensities of the reflections are indicated in Table 3. The relative intensity indicates the measured intensity of the given line as a percentage of the intensity of the (211) reflection. In the “isotropy” column, the relative intensities of the reflections for pulverized samples are indicated, in accordance with the JCPDS.

The “orientation” column indicates the approximate orientation of a given lattice plane relative to the c-axis.

### EXAMPLE 3

153 g Ca(NO₃)₂—4H₂O are dissolved in 1 l aqua bidest (18 MΩ/cm). 250 ml of this are drawn off and mixed with 44 g NH₃ (32%). 17.33 g (NH₄)₂HPO₄ are dissolved in 1 l aqua bidest (18MΩ/cm). 750 ml of this are drawn off and mixed with 8.8 g NH₃ (32%). All chemicals used possess the purity level p.a. To the receiving flask, 1.1 l aqua bidest, 30 ml of the Ca solution, and 8.8 g NH₃ (32%) are added, and the contents are heated to 80° C. The reaction takes place in an external reaction vessel that has a volume of ca. 5 ml, a throughput rate of ca. 78 ml/s, and a stirring speed of 160/s, at a constant temperature. The Ca solution is added to the receiving flask dropwise, at a rate of ca. 0.33 ml/s. The phosphate solution is introduced into the external reaction vessel at a rate of 0.83 ml/s.

Upon completion of the reaction, the precipitate is allowed to rest on the mother liquor for 18 h at 60° C. (with agitation at 100 min⁻¹), after which it is washed with room temperature aqua bidest until the nitrate level in the rinsing water is <20 ppm. Following filtration and drying at 210° C., a yield of 14 g precipitate is obtained. The precipitate is comprised of elongated, lusterless crystallites, whose length ranges between 150 nm and 400 nm, and whose thickness ranges between 50 nm and 120 nm; see FIG. 3.

For further processing, the precipitate is ground in an agate mortar to particles that are <250 μm, is axially pressed at 800 bar, and is then sintered using the following time/temperature profile: room temperature up to 400° C.: 13° C./min; stationary 400° C.: 60 min; 400° C. to 850° C.: 10° C./min; stationary 850° C.: 120 min; 850° C. to 1195° C.: 3° C./min; stationary 1195° C.: 60 min; cooling to room temperature: ca. 1.5° C./min.

The result of the sintering is a translucent body having a thickness of 3.14 g/cm³. The double refraction was calculated as Δn=(1.1±0.2)*10⁻³, with the c-axis being perpendicular to the pressing direction. The x-ray diffraction diagram indicates that the sintered body is pure hydroxylapatite.

The rod-shaped form of the monocrystallites obtained in the three examples can be identified using a scanning electron microscope and via x-ray diffraction. FIG. 1 shows a scanning electron microscope image of the calcium phosphate precipitated in accordance with the procedures in Example 1, enlarged 30,000 times. Here the individual particles appear as elongated crystallites with dimensions of ca. 150 nm by 50 nm. The x-ray diffraction diagram shows the needle-like character of the precipitated crystallites more clearly. Table 1 gives the half-intensity width of the lines of the precipitation of the calcium phosphate precipitated in accordance with Example 1. A comparison of the line width of the (002) reflection, narrower by a factor of 2, whose lattice planes are perpendicular to the c-axis, and the (200)-reflection, whose lattice planes lie parallel to the c-axis, accentuates the needle-like form of the crystallites.

A dental prosthesis made from this sintered material will be natural looking and stable within the oral environment. In terms of demineralization and remineralization it will behave essentially like natural tooth enamel.

TABLE 1
Half-Intensity Width of the Lines in 2*Θ

| Reflection | Example 1 |
|---|---|
| (002) | 0.156 |
| (102) | 0.223 |
| (111) | 0.242 |
| (200) | 0.334 |
| (202) | 0.408 |
| (211) | 0.431 |
| (310) | 0.491 |
| (210) | 0.384 |
| (301) | 0.912 |
| (300) | 0.601 |
| (212) | 0.596 |

TABLE 2

| | Line | | Relative | | |
|---|---|---|---|---|---|
| Lattice Plane | Width | Intensity | Intensity | Isotropy | Orientation |
| (002) | 0.084 | 9.69 | 7 | 40 | ⊥ |
| (112) | 0.092 | 42.97 | 32 | 60 | Inclined |
| (200) | 0.049 | 8.26 | 6 | 10 | ∥ |
| (210) | 0.054 | 29.87 | 22 | 17 | ∥ |
| (211) | 0.062 | 135.44 | 100 | 100 | |
| (300) | 0.066 | 128.97 | 95 | 60 | ∥ |
| (310) | 0.075 | 49.38 | 37 | 20 | ∥ |

TABLE 3

| | Line | | Relative | | |
|---|---|---|---|---|---|
| Lattice Plane | Width | Intensity | Intensity | Isotropy | Orientation |
| (002) | 0.051 | 7.09 | 5 | 40 | ⊥ |
| (112) | 0.061 | 26.36 | 18 | 60 | Inclined |
| (200) | 0.046 | 9.08 | 6 | 10 | ∥ |
| (210) | 0.087 | 53.65 | 37 | 17 | ∥ |
| (211) | 0.063 | 145.71 | 100 | 100 | |
| (300) | 0.065 | 142.64 | 98 | 60 | ∥ |
| (310) | 0.071 | 65.52 | 45 | 20 | ∥ |

1. Dental ceramic containing a share of more than 90% by weight hydroxylapatite (HA; Ca₅(PO₄)₃OH), characterized in that the ceramic is anisotropic.

2. Dental ceramic according to claim 1, characterized in that the refraction index is anisotropic within the spectrum of visible light, and the green body and/or the sintered body exhibit double refraction.

3. Dental ceramic according to one of the preceding claims, characterized in that the difference in the refraction indices is Δn>1×10⁻⁴, especially Δn>2×10⁻³.

4. Dental ceramic according to one of the preceding claims, characterized in that the sintered body is anisotropic with respect to x-ray diffraction, wherein the intensity of reflections resulting from textural effects are changed by preferred directions in the sintered body.

5. Dental ceramic according to one of the preceding claims, characterized in that the anisotropy is oriented perpendicular to a given axis.

6. Dental ceramic according to one of the preceding claims, characterized in that the content of tricalcium phosphate (TCP; Ca₃(PO₄)₂) and/or another poorly soluble phosphate is less than or equal to 4%.

7. Process for manufacturing a dental ceramic, comprising the following steps:
Precipitation of at least one
Calcium phosphate compound from an aqueous or organo-aqueous solution to form a precipitate;
If necessary, washing, drying, and possibly grinding of the precipitate;
Pressing of the precipitate to form a green body;
Sintering of the green body;
characterized in that the Ca/P atomic ratio lies between 1.66 and 1.68.

8. Process according to claim 7, characterized in that the calcium phosphate compound is substantially stoichiometric HA.

9. Process according to one of the preceding claims, characterized in that the pressing of the green body is implemented at an intrinsic pressure of 200 bar to 10,000 bar, preferably 800 bar to 1500 bar.

10. Process according to one of the preceding claims, characterized in that the pressing is implemented in an axial direction.

11. Process according to one of the preceding claims, characterized in that the pressing is implemented in an axial direction using an extrusion die, wherein the extrusion die is rotated around its axis.

12. Dental ceramic, produced via a process as specified in one of the preceding claims 7 through 11.

13. Crystalline hydroxylapatite as the starting material for use in dental applications, characterized in that the crystals are rod-shaped, and measure 70 nm to 1000 nm in length and between 7 nm and 500 nm in thickness.
